(19) United States
(12) Patent Application Publication
(10) Pub. No.: US 2025/0078973 A1
(43) Pub. Date: Mar. 6, 2025

(54) DECISION SUPPORT AUTHORIZING ENVIRONMENT FOR CLINICAL DECISION SUPPORT SYSTEM

(71) Applicant: Laboratory Corporation of America Holdings, Burlington, NC (US)

(72) Inventors: Enno DE VRIES, Burlington, NC (US); Jennifer ENNIS, Chicago, IL (US); Susan DONAHUE, Chicago, IL (US); Aymeric GRASSART, Chicago, IL (US); David KAWLICHE, Culver, IN (US); Melissa ALEXANDER, Chicago, IL (US)

(21) Appl. No.: 18/804,563

(22) Filed: Aug. 14, 2024

Related U.S. Application Data
(60) Provisional application No. 63/579,476, filed on Aug. 29, 2023.

(51) Int. Cl. G16H 20/00; G16H 10/40; G16H 10/60; G16H 15/00

(52) U.S. Cl. CPC G16H 20/00; G16H 10/40; G16H 10/60; G16H 15/00

(57) ABSTRACT

A method can involve receiving input data relevant to a patient encounter such that the input data may be compliant with a standard for exchanging healthcare information. The method may involve determining a subset of functions from a plurality of functions based on parameters associated with the patient encounter, conditions met by the input data, or a consuming system that received a customized report and for each function in the subset of functions, the method may perform at least one transformation on the input data such that performing the at least one transforming comprises producing an assigned dynamic value from among the plurality of dynamic values associated with the function. The method may involve automatically generating the customized report for the patient encounter based on the assigned dynamic values from each function in the subset of functions and transmitting an order request to order a lab test.

### CROSS-REFERENCE TO RELATED APPLICATION

This claims the benefit of priority to U.S. Provisional Application No. 63/579,476, filed Aug. 29, 2023, the entire contents of which are incorporated by reference herein for all purposes.

### FIELD

The present disclosure relates generally to medical records systems, and more particularly, to a computer-based authoring environment for a clinical decision support system.

### BACKGROUND

In the medical field, treatments of diseases can be challenging, especially when a disease is complex. Medical service providers can have a limited window of time in which to make critical decisions regarding patient treatment and care during a patient encounter. When dealing with a complex medical condition, the medical provider may be faced with a voluminous amount of information from which to determine an effective course of treatment. Yet a situation of the patient may be such that the medical provider does not know or does not have time to adequately review all of the relevant information regarding the effective course of treatment. Clinical decision support (CDS) computing systems can assist the medical provider with determining the effective course of treatment. Designing, editing, and updating CDS computing systems using conventional methods of computer programming can be time consuming and labor intensive.

### BRIEF SUMMARY

Techniques disclosed herein relate generally to medical records systems. More specifically and without limitation, techniques disclosed herein relate to an authoring environment for a clinical decision support system that can provide tools, information, and guidance to a medical practitioner at a point of care regarding a patient.

In accordance with aspects of the present disclosure, a method may include receiving, by a computing device, input data relevant to a patient encounter where the input data may be compliant with a standard for exchanging healthcare information. The method may further include determining, by the computing device, a subset of functions from a plurality of functions based on parameters associated with the patient encounter, conditions met by the input data, or a consuming system that receives a customized report. The method may further include, for each function in the subset of functions, performing, by the computing device, at least one transformation on the input data such that performing the at least one transformation may include producing an assigned dynamic value from among a plurality of dynamic values associated with the function and automatically generating, by the computing device, the customized report for the patient encounter based on the assigned dynamic values from each function in the subset of functions. In some examples, the method may include transmitting an order request, by the computing device, to order a lab test based, at least partially, on the customized report or the assigned dynamic values from each function in the subset of functions and in response to receiving a response to the order request, automatically transmitting, by the computing device, the order request to an automated test ordering system to perform the lab test.

In accordance with aspects of the present disclosure, the consuming system may be a diagnostic assistant tool application. In some embodiments, automatically generating the customized report comprises may include automatically generating a graphical user interface (GUI) within the diagnostic assistant tool application and incorporating the customized report within the GUI.

In accordance with aspects of the present disclosure, the method may include developing each function in the plurality of functions in an authoring environment.

In accordance with aspects of the present disclosure, a portion of functions in the subset of functions may be associated with at least one plan description defined in the authoring environment.

In accordance with aspects of the present disclosure, developing each function in the plurality of functions may include developing, in the authoring environment, each function to include sets of rules, logics, matrix logic, other common functions, artificial intelligence (AI) models, or value sets.

In accordance with aspects of the present disclosure, developing each function in the plurality of functions may include accessing, in the authoring environment, at least a portion of the sets of rules, logics, matrix logic, other common functions, AI models, or value sets from a library.

In accordance with aspects of the present disclosure, developing each function in the plurality of functions may include developed each function using clinical quality language (CQL) programming code.

In accordance with aspects of the present disclosure, the method may include receiving, by the computing device, a customized spreadsheet for at least one function developed in the authoring environment and automatically converting, by the computing device, the customized spreadsheet into the CQL programming code.

In accordance with aspects of the present disclosure, a clinical decision support (CDS) system may include one or more processors and one or more memories that include instructions executable by the one or more processors to perform operations. In some examples, the operations may include receiving input data relevant to a patient encounter such that the input data may be compliant with a standard for exchanging healthcare information. The operations may further include determining a subset of functions from a plurality of functions based on parameters associated with the patient encounter, conditions met by the input data, or a consuming system that received a customized report. The operations may further include, for each function in the subset of functions, performing, by the computing device, at least one transformation on the input data such that performing the at least one transformation may include producing an assigned dynamic value from among a plurality of dynamic values associated with the function and automatically generating, by the computing device, the customized report for the patient encounter based on the assigned dynamic values from each function in the subset of functions. In some examples, the operations may include transmitting an order request, by the computing device, to order a lab test based, at least partially, on the customized report or the assigned dynamic values from each function in the subset of functions and in response to receiving a response to the order request, automatically transmitting, by the computing device, the order request to an automated test ordering system to perform the lab test.

In accordance with aspects of the present disclosure, the consuming system may be a diagnostic assistant tool application such that the operation of automatically generating the customized report may include automatically generating a graphical user interface (GUI) within the diagnostic assistant tool application and may incorporate the customized report within the GUI.

In accordance with aspects of the present disclosure, the operations may include developing each function of the plurality of functions in an authoring environment.

In accordance with aspects of the present disclosure, a portion of functions in the subset of functions may be associated with at least one plan description defined in the authoring environment.

In accordance with aspects of the present disclosure, the operation of developing each function in the plurality of functions may include developing, in the authoring environment, each function to include sets of rules, logics, matrix logic, other common functions, artificial intelligence (AI) models, or value sets.

In accordance with aspects of the present disclosure, the operation of developing each function in the plurality of functions may include accessing, in the authoring environment, at least a portion of the sets of rules, logics, matrix logic, other common functions, AI models, or value sets from a library.

In accordance with aspects of the present disclosure, the operation of developing each function in the plurality of functions may include developing each function using clinical quality language (CQL) programming code.

In accordance with aspects of the present disclosure, the operations may include receiving, by the computing device, a customized spreadsheet for at least one function developed in the authoring environment and automatically converting, by the computing device, the customized spreadsheet into the CQL programming code.

In accordance with aspects of the present disclosure, a non-transitory computer-readable medium may include instructions that are executable by one or more processing devices for causing the one or more processing devices to perform operations that may include receiving input data relevant to a patient encounter, the input data compliant with a standard for exchanging healthcare information and determining a subset of functions from a plurality of functions based on parameters associated with the patient encounter, conditions met by the input data, or a consuming system that received a customized report. The operations may further include, for each function in the subset of functions, performing, by the computing device, at least one transformation on the input data such that performing the at least one transformation may include producing an assigned dynamic value from among a plurality of dynamic values associated with the function and automatically generating, by the computing device, the customized report for the patient encounter based on the assigned dynamic values from each function in the subset of functions. In some examples, the operations may include transmitting an order request, by the computing device, to order a lab test based, at least partially, on the customized report or the assigned dynamic values from each function in the subset of functions and in response to receiving a response to the order request, automatically transmitting, by the computing device, the order request to an automated test ordering system to perform the lab test.

In accordance with aspects of the present disclosure, the consuming system may be a diagnostic assistant tool application and wherein the operation of automatically generating the customized report comprises automatically generating a graphical user interface (GUI) within the diagnostic assistant tool application and incorporating the customized report within the GUI.

In accordance with aspects of the present disclosure, the operations may include developing each function of the plurality of functions in an authoring environment.

In accordance with aspects of the present disclosure, a portion of functions in the subset of functions may be associated with at least one plan description defined in the authoring environment.

In accordance with aspects of the present disclosure, the operation of developing each function in the plurality of functions comprises developing, in the authoring environment, each function may include sets of rules, logics, matrix logic, other common functions, artificial intelligence (AI) models, or value sets.

In accordance with aspects of the present disclosure, the operation of developing each function in the plurality of functions further may include accessing, in the authoring environment, at least a portion of the sets of rules, logics, matrix logic, other common functions, AI models, or value sets from a library.

In accordance with aspects of the present disclosure, the operation of developing each function in the plurality of functions may include developing each function using clinical quality language (CQL) programming code.

In accordance with aspects of the present disclosure, the operations may include receiving, by the computing device, a customized spreadsheet for at least one function developed in the authoring environment and automatically converting, by the computing device, the customized spreadsheet into the CQL programming code.

### BRIEF DESCRIPTION OF THE DRAWINGS

FIG. 1 is a flow diagram of a flow of information through a clinical decision support (CDS) system according to some embodiments of the present disclosure.

FIG. 2 is a block diagram of a function for a clinical decision support (CDS) engine that can receive data, perform actions on data, and produce Fast Healthcare Interoperability Resources (FHIR) outputs according to some embodiments of the present disclosure.

FIG. 3 is a diagram of graphical icons for tools for an authoring environment for a CDS system according to some embodiments of the present disclosure.

FIG. 4 is a flow chart of an example of a process for automatically generating a customized report for a patient encounter according to some embodiments of the present disclosure.

FIG. 5 is a depiction of a graphical user interface (GUI) within an authoring environment for a CDS system according to some embodiments of the present disclosure.

FIGS. 6A-6B form a diagram showing characteristics of customized reports generated by a CDS system according to some embodiments of the present disclosure.

FIGS. 7A-7G form a schematic of customized reports tailored to consuming systems by a CDS system according to some embodiments of the present disclosure.

FIG. 8 is block diagram of a computing device for use with the CDS system in accordance with various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Conventionally, in healthcare fields such as medicine, treatment, diagnosis or similar, treatments of diseases are becoming more challenging to identify with advances in new techniques, medicine, and treatment programs, especially when a disease is complex. Medical professionals may have a limited window of time in which to make critical decisions regarding patient treatment and care during a patient encounter. When dealing with a complex medical condition, the medical professional may be faced with more information that may be able to be processed during the limited window of time. This makes determining an accurate and precise effective course of treatment in the limited window of time challenging. Additionally, a situation of the patient may be such that the medical provider does not know or does not have time to adequately review all of the relevant information regarding the effective course of treatment during the patient visit and may need to take time outside of normal hours to produce a viable, patient oriented, customized report. Clinical decision support (CDS) computing systems can assist the medical provider with determining the effective course of treatment. Moreover, designing, editing, and updating CDS computing systems using conventional methods of computer programming can be time consuming, inaccurate, unprecise, utilize stale data, and may be data and labor intensive.

Certain aspects and features of the present disclosure relate to an authoring environment for a clinical decision support (CDS) system. The CDS system can provide improved tools, a technically improved and patient catered report that may include accurate and precise information, and guidance to a medical practitioner at a point of care regarding a patient. The CDS system can receive patient data as input for a CDS engine. The CDS engine can perform a series of actions and/or transformations (e.g., convolutions, cross-correlations) to the data and produce outputs. The outputs can be used to automatically generate a comprehensive report that can be shared with the medical practitioner at the point of care. The authoring environment allows users to add, edit, or control the series of actions and/or transformations performed by the CDS engine. The technical field of generating customized comprehensive reports is substantially improved by the authoring environment by aiding in customization, catering to individual patients needs, and removing some or all information that may not be relevant to the patient by allowing users to add, edit, or control the series of actions and/or transformations performed by the CDS engine.

The series of actions can be performed by programmed cells or functions of the CDS engine. Each function can include action parameters such as logic, matrices, equations, medical principles, trained artificial intelligence (AI) (or machine-learning (ML)) models, sets of rules, etc. that can be used to help perform the series of actions to data inputs. The action parameters can be defined for the functions within the authoring environment. In some examples, action parameters can be described using computer programming languages, such as clinical quality language (CQL), and some complex action parameters using computer programming language. Conventionally, programming in computer programming languages can be time-consuming and labor intensive to generate, revise, or otherwise manage, etc. and may not account for a particular disease, treatment, or unique plan created by a medical professional. The technical field of medical programming is improved by the CDS engine's authoring environment which may implement several tools that can drastically reduce time spent by an entity producing computer code for components of the CDS engine to generate customized reports for particular treatments, diseases, or plans. The entity can include personnel, such as a computer programmer. For example, the authoring

In addition to the aforementioned technical improvements, the authoring environment can include a library tool. From the library tool, an entity can access a database with pre-developed functions that the entity can incorporate in a development of a new function. Instead of writing many lines of code to develop the new function, the entity can combine and edit pre-developed functions, saving a considerable amount of time and labor. Additionally or alternatively, the authoring environment can include an accelerators tool. The accelerators tool can provide the entity with access to accelerators.

Accelerators can be customized spreadsheets, such as program and discipline-specific authoring spreadsheets. The authoring spreadsheets can describe rules and functions that can be imported and automatically converted into CQL code. Accelerators can allow for large, complex, and repetitive parameter sets, such as parameter sets associated with NGS. Accelerators can also permit simple comma-separated values (CSV) file imports of third-party rule sets that can automatically be converted into CQL code. The authoring environment can also include a code system tool. The code system tool can include a real-time updated dictionary of terms, such as medical terms that can be used to automatically update the dynamic values of functions. The authoring environment can have a graphical user interface (GUI) that includes icons for several tools, such as the accelerator tool and the library tool. A user can easily gain access to features of a tool by selecting an associated icon. Via the accelerator tool, the authoring environment for the CDS system can provide a way to produce complex functions with a very large set of rules or dynamic values by describing the complex functions in spreadsheet formats that can automatically be converted into computer code such as CQL code by the CDS system. Creating the complex functions in this manner can reduce an amount of resources involved in producing the complex functions. Conventionally, medical programming, and specifically computer programming, have been largely inaccessible to those unskilled in the specifics of the particular language chosen and provide few or no alternatives to a user to edit, update, or create functions and/or custom outputs. The present disclosure provides a significant technical improvement in the field of medical programming in that the accelerator tool can also provide a way for anyone, not just skilled software developers, to customize, edit, or create functions for the CDS engine that have certain features without having to perform any software coding. The CDS system can also receive computer programs in any computing language or format, such as CSV format, from third parties and convert the computer programs into programs with CQL code.

In addition, the CDS engine may provide a decision support authoring environment for creating, managing, and deploying CDS processes (e.g., code systems, value sets, etc.) for various clinical domains and applications. The CDS engine provides clinicians, patients, and other stakeholders (e.g., administration, nurses, etc.) with timely and relevant information and guidance to enhance health and health care. The CDS engine may help improve the quality, safety, efficiency, and effectiveness of health care delivery by providing evidence-based recommendations, alerts, reminders, order sets, care plans, and other forms of assistance. Unlike conventional medical systems, the CDS engine improves the technical field by also supporting patient engagement, education, and self-management by providing personalized and tailored information and feedback based on the functions associated with the patient. In addition to the aforementioned technical improvements, the CDS engine may be interoperable, scalable, and adaptable to different clinical settings, workflows, and user preferences. Moreover, the CDS engine enables timely updates that are validated regularly to reflect the latest scientific evidence, clinical guidelines, and best practices. Utilizing the CDS engine enables the creation, management, and deployment of CDS processes for various clinical domains and applications using a standardized, ease-to-understand, and expressive functional language, a comprehensive and extensible terminology service, and a flexible and modular deployment service.

In some examples, the decision support authoring environment may include an authoring tool for creating, editing, testing, and validating CDS processes such as code systems, value sets, plan definitions, and CQL expressions. The authoring tool may allow users to define the logic, data, and actions of the CDS processes, using the GUI or a text editor. The authoring tool may also support import and export of CDS processes in various formats such as, but not limited to, Fast Healthcare Interoperability Resources (FHIR), Health Level Seven (HL7), and Clinical Quality Framework (CQF). The decision support authoring environment may additionally include a terminology service. The terminology service may be used for managing, mapping, and resolving clinical concepts, codes, and terms. The terminology service may provide access to a variety of terminologies, vocabularies, and ontologies such as Systemized Nomenclature of Medicine—Clinical Terms (SNOMED CT), Logical Observation Identifiers Names and Codes (LOINC), RxNorm, International Classification of Diseases (ICD), and Unified Medical Language System (UMLS). The terminology service may also support the creation and/or maintenance of custom terminologies and/or value sets, as well as the alignment and harmonization of different terminologies and code systems. This support substantially improves the flexibility and adaptability of the CDS engine by enabling real-time updates to terminology for a user catered experience.

In some examples, the decision support authoring environment may include a deployment service for publishing, distributing, and executing the CDS processes across different clinical applications and platforms. The deployment service may provide application programming interfaces (APIs) for integrating CDS processes with various systems such as electronic health records (EHRs), laboratory information systems (LISs), patient portals, mobile apps, and reporting dashboards. In some examples, the deployment service may also support the configuration and/or customization of CDS processes according to the specific needs and preferences of the end-users, such as clinicians, patients, researchers, or administrators. In some non-limiting examples, the decision support authoring environment may be used to create, manage, and deploy CDS processes for various clinical domains and applications such as chronic kidney disease, cervical risk management, oncology DNA sequencing, diagnostic outcome reporting, patient handouts, and disease CDS programs. The decision support authoring environment may also be used to create, manage, and deploy CDS processes for various diseases and delivery methods to significantly improve screening, monitoring, treatment suggestions, guidance on lifestyle modifications, medication adherence, self-management, and/or education for domains such as diabetes, cardiovascular, toxicology, autoimmune diseases, and oncology.

In some examples, the CDS engine may automatically order lab tests based on the customized report. Conventionally, medical professionals would need to schedule lab tests based on various factors such as diagnosis, disease, or similar, and as discussed above, the medical professionals may not have enough time during the time window to accurately diagnose the disease. The CDS engine may improve the technical field of lab test ordering for patients by identifying values within the customized report and automatically ordering lab tests based on those values. This process does not exist in conventional systems. The CDS engine may identify which tests are valid to the patient and check networked systems to schedule the lab test.

The following illustrative examples are presented to introduce the reader to the general subject matter discussed herein and are not intended to limit the scope of the disclosed concepts. The following sections describe various additional features and examples with reference to the drawings in which like numerals indicate like elements and directional descriptions are used to describe the illustrative aspects but, like the illustrative aspects, should not be used to limit the present disclosure. Additionally, the presented figures are generally described with respect to medical techniques and subject matter, but the general subject matter discussed herein is not limited to medical techniques and subject matter.

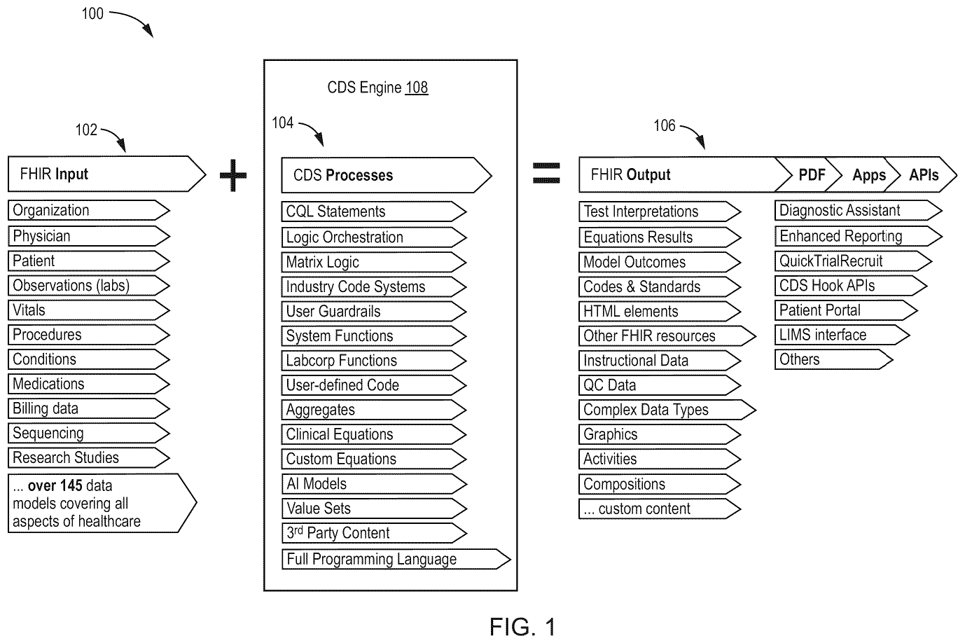

FIG. 1 is a flow diagram of a flow of information through a CDS system 100 according to some embodiments of the present disclosure. The flow of information can occur during a patient encounter with a user of the CDS system 100. The user can be a medical practitioner (e.g., nurse, doctor, psychiatrist, etc.). The CDS system 100 can receive data in the form of Fast Healthcare Interoperability Resources (FHIR) input 102. FHIR can define a standard for exchanging healthcare information electronically. Data included in the FHIR 102 can be configured to meet the FHIR standard. The data can be received in any form of programming language, including Hypertext Markup Language (HTML) or JavaScript Object Notation (JSON). The data can be associated with the patient who is visiting the user.

Forms of data included in the FHIR input 102 can include organizational data, physician data, patient data, observational data related to the patient, vitals for the patient, procedures, medical conditions, medications, billing data, genome sequencing data, research studies relevant to the patient, etc. The observational data can include any combination of laboratory results including test results, test panel results, test chemistry results, analytic results, diagnostic procedure results, etc. Additionally, observational data can include various levels of details depending on types of tests performed and information being conveyed. For example, observational data can include laboratory results, physiological results, mental results, physical results, etc.

The FHIR input 102 can be received from a collection of databases. A record management system can aggregate and deduplicate data from the collection of databases to form the FHIR input 102 before transmitting the FHIR input 102 to the CDS system 100. The collection of databases can include data from multiple disjunctive sources. The multiple disjunctive sources can include a combination of electronic health record (EHR) systems, electronic medical record (EMR) systems, outpatient facilities, such as testing facilities, any medical facility visited by the patient, and any other suitable disjunctive sources.

The FHIR input 102 can be considered input data for CDS processes 104. Each process in the CDS processes 104 can be performed by a CDS engine 108. The CDS engine 108 can be programmed to execute the CDS processes 104 via instructions in various forms of programming language. A full programming language associated with the CDS processes 104 can include clinical quality language (CQL) statements, logic orchestration, matrix logic, industry code systems, user guardrails, system functions, functions produced by a server operator, user-defined code, aggregates, clinical equations or third-party equations, custom equations, artificial intelligence (AI) models, value sets, third party content, etc. CQL can be a computer language code customized to clinical decision aspects of medical care. The system functions can be health care specific. Aggregates can include processes that assist in determining occurrences of multiple diseases. User guardrails can describe an integrated development environment for industry code systems, logic, etc. The custom equations can include equations developed by AI models that can involve converting medical journal text into equation form. Functions of the CDS processes 104 can receive data in the FHIR input 102 as inputs, perform actions and/or transformations (e.g., convolutions, cross-correlations) on the data, and produce FHIR outputs 106.

The functions can pre-filter the FHIR input 102 prior to performing the actions and/or transformations. For example, a function can filter out observational data with a date stamp prior to 2019, or any other suitable year or time. Conversely, the function can select, based on data requirements built into the function, the last seven laboratory results for the patient as input data. A function can include dynamic values. When the function performs actions and/or transformations on input data, the dynamic values can become assigned. Dynamic values can include sentences, sentence fragments, multiple sentences, graph values, graphical elements, instructions, and the like. Prior to performing an action, the function can have many possible dynamic values. After performing the action, the function can have a single assigned dynamic value or several assigned dynamic values. The FHIR outputs 106 produced by the functions can be based on assigned dynamic values.

The CDS engine 108 can include various functions. For each patient encounter, a subset of functions can perform actions on the FHIR input 102 and produce assigned dynamic values. Functions of the CDS engine 108 that are not included in the subset of functions may not perform actions on the FHIR input 102. The CDS engine 108 can evaluate the FHIR input and determine which functions to include in the subset of functions. Each function can include metadata. The metadata can include a category for the function. The category can describe a disease, clinical group, etc. that is associated with the function. For example, the metadata for a function can indicate that the function is associated with a cardiovascular clinical group.

The FHIR outputs 106 can be collected, organized and presented to a user via a consuming system. Examples of consuming systems can include a diagnostic assistant tool application, an application for evaluating potential clinical trials for the patient, application programming interfaces (APIs), patient portals, laboratory information management systems (LIMS), various interfaces, and the like. The FHIR outputs 106 can include text interpretations, equation results, model outcomes, codes and standards, html elements, other FHIR resources, instructional data, QC data, complex data types, graphics, activities, compositions, and custom content.

The CDS system 100 can present the FHIR outputs 106 by generating a customized report for the user. The customized report can be populated by the assigned dynamic values. Assigned dynamic values can include or describe a sentence, sentence fragment, multiple sentences, paragraphs, graph values, graphical elements like figures, and the like to be included in the customized report. A form of the customized report can depend on the consuming system. The customized report can take the form of a portable document format (PDF) file, an application, an API, etc. In addition to generating customized reports, the CDS system 100 can transmit instructions to a device based on the FHIR outputs 106. For example, the FHIR outputs 106 can include an interactable recommendation (e.g., clickable button, hyperlink) to order a particular lab test for the patient (e.g., blood work, glucose check). For example, the interactable recommendation may include one or more fields that may be clicked on, or otherwise interacted with, and approved, rejected, scheduled, and/or postponed by a medical professional to order and/or cancel a lab test. The CDS system 100 can instruct, based on assigned dynamic values or the customized reports included in the FHIR outputs 106, a device to automatically transmit an order request to an automized test ordering system. Prior to transmitting the order request, the user or another medical practitioner can review and approve the order request. In some examples, the medical professional may supplemental and/or replace the recommended lab test by manually adjusting (e.g., using the authoring environment) the customized report.

The customized report can be specified and customized for a disease, condition, medical department, clinical group, etc. The CDS system 100 can identify a disease, condition, medical department, clinical group, etc. to associate the customized report with based on the metadata of functions in the subset of functions that perform actions and/transformations on the FHIR input 102. In some examples, the CDS system 100 can identify more than one disease, condition, medical department, clinical group, etc. The CDS system 100 can recommend courses of action to address a condition, such as treatment suggestions based on known public guidelines, expert opinions, etc. The customized report can include precision medicine driven content. The CDS system 100 via the CDS engine 108 can generate several customized reports, one for each disease, condition, medical department, clinical group, etc. that is identified.

In some examples, the customized report may include details for providing patient care related to conditions including, but not limited to, allergies (e.g., dog dander, fish, wheat, etc.), cardiac levels (e.g., total cholesterol, high-density lipoproteins (HDL), low-density lipoproteins (LDL), etc.), endocrine levels (e.g., thyroid stimulating hormone (TSH), ovarian reserve, etc.), general medicine values (e.g., calcium levels, glucose levels, A:G ratio, etc.), gastrointestinal (GI) data (e.g., celiac disease data, tissue transglutaminase data (tTG), etc.), hematology values (e.g., iron levels, hemoglobin, platelet counts, etc.), fecal occult blood test data, infectious disease data (e.g., varicella, chlamydia, rubella), or urine studies (e.g., albumin levels, ketone levels, bacteria, etc.).

The CDS system 100 can include an authoring environment. Within the authoring environment, the entity can add, remove, or edit the CDS processes 104 of the CDS engine 108. The entity can include administrators, computer scientists, programmers, medical department heads, laboratory directors, authorized users, etc. The entity can add or remove new functions, edit components of existing functions, update programming languages used to define functions, and apply accelerators to reduce an amount of resources involved in producing certain functions such as large or complex functions.

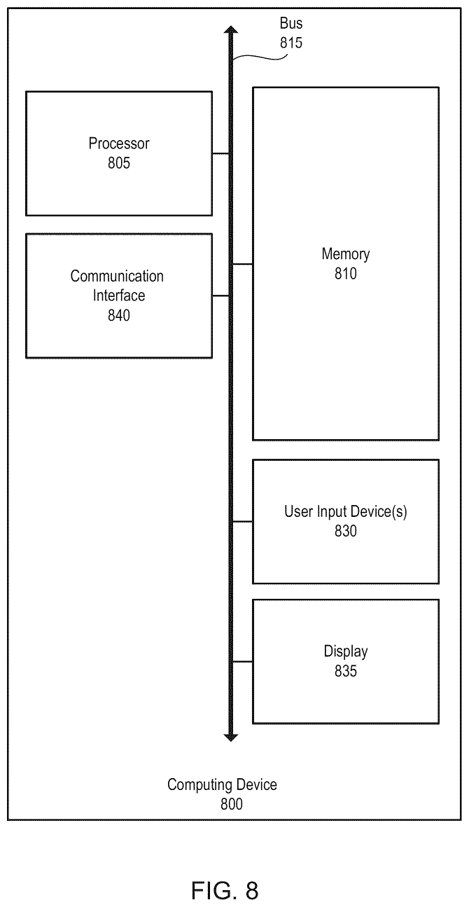

In some examples, the AI models may function to store calculated values for one or more of the functions and/or CDS processes 104 in memory (e.g., memory 810 of FIG. 8) for later retrieval. For example, if a library of literature pertaining to gene variant rules, results, risks, interpretations, or tables is assessed by the AI models, the AI models may update existing corresponding functions and/or CDS processes 104 in a feedback loop type scheme. In some examples, the AI models may apply labels to some or all of the functions and/or CDS processes 104 as supervised or unsupervised data in order to train and learn from new customized functions and/or CDS processes 104 created and/or updated by the patient and/or user of the CDS system 100. In a non-limiting example, a medical professional may like to include descriptions of specific diseases with each report to help the patient understand the diagnosis and have literature to review upon diagnosis. Other medical professionals may add personalized outputs to the reports to provide better “bedside manner” to patients with complex diagnosis to help foster a stronger patient-medical professional relationship.

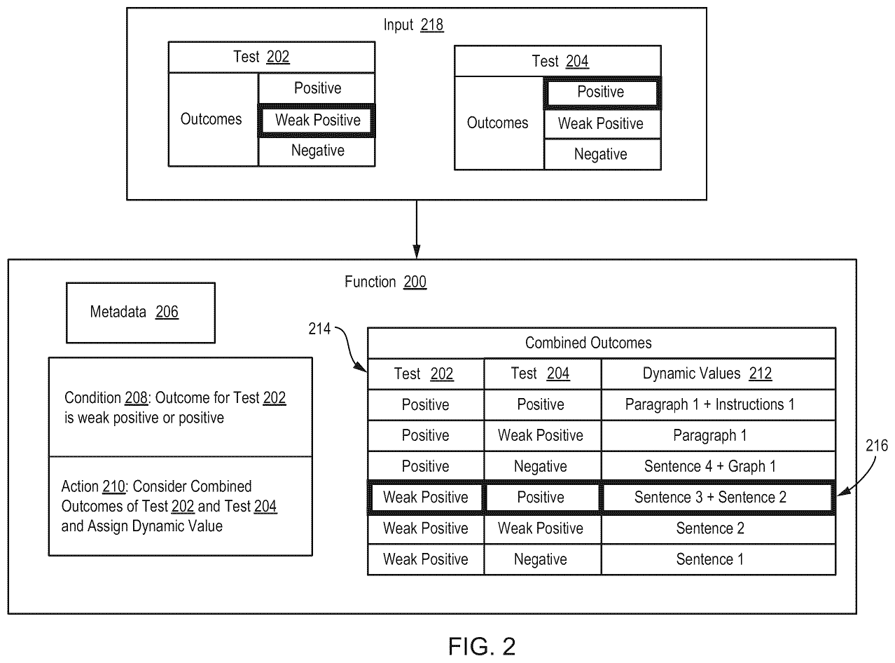

FIG. 2 is a block diagram of a function 200 for a CDS engine 108 that can receive data in a form of an input 218, perform actions on the data, and assign dynamic values for inclusion in FHIR outputs 106 according to some embodiments of the present disclosure. The function 200 can receive as the input 218 results of two laboratory tests, Test 202 and Test 204. Functions for the CDS engine 108 can include components, such as metadata 206, a condition 208, an action 210, an evaluation matrix 214, and dynamic values 212. Although the function 200 in FIG. 2 includes a single action 210, a single condition 208, and a single evaluation matrix 214; functions of the CDS engine 108 can include any number of actions, conditions, and evaluation matrices. The metadata 206 can include information that categorizes the function 200 and describes pre-filter steps to apply to data in the input 218. The metadata 206 can indicate diseases, conditions, medical departments, clinical groups, etc. to associate with the function 200.

A result for each laboratory test can indicate a presence of a medical condition or feature, such as a disease or an abnormal medical status. In some examples, the medical condition can be difficult to diagnose and can include several symptoms. Examples of such conditions can include chronic kidney disease, diabetes, cardiovascular disease, and the like. In some embodiments, Test 202 and Test 204 can test for different indicators of a single disease. Each of the two laboratory tests can have three possible distinct outcomes. For example, the outcomes for Test 202 can include a positive result, a weak positive result, or a negative result. Similarly, outcomes for Test 204 can include a positive result, a weak positive result, or a negative result.

The function 200 can receive results of both Test 202 and Test 204 as the input 218, perform the action 210 (e.g., evaluate the two results in combination) on the input 218, and produce an assigned dynamic value 216 from among the dynamic values 212 associated with the function 200. The assigned dynamic value 216 can be included in FHIR outputs 106 associated with a patient encounter. In some examples, the function 200 will not perform the action 210 and the dynamic values 212 can remain unassigned and may not be included in FHIR outputs 106. The CDS engine 108 can confirm that the patient has received results for both Test 202 and Test 204. If the patient has not received results for either of Test 202 or Test 204, the function 200 may not receive the results as the input 218 and may not perform the action 210 to the results. If the patient has received both test results, the function 200 can receive the two test results as the input 218.

In some examples, data within the input 218 can be pre-filtered before being received by the function 200. For example, if Test 202 or Test 204 has been administered to a patient multiple times, so that the patient has received multiple results for either or both tests, the multiple results in the input 218 can be pre-filtered. The function 200 may only receive a most recent result, a most common result, or an average result for each test as the input 218. For example, data associated with the patient can include ten results for Test 202 and three results for Test 204. The input 218 can include a most common result of five most recent results for Test 202 and an average of assigned values for all results for Test 204. The last ten results for Test 202, from oldest to newest, can include: negative, negative, negative, negative, negative, negative, weak positive, negative, weak positive, and weak positive. The results for Test 202 can include weak positive, positive, and positive with assigned values of 0 for negative, 1 for weak positive, and 3 for positive. In this example, the input 218 would include a result of weak positive for Test 202 (since weak positive occurred three times out of the last five results for Test 202) and positive for Test 204 (average for the results of Test 204 is 2.33, which is closer to 3 (positive) than to 1 (weak positive)).

The condition 208 of the function 200 can determine if the action 210 of the function 200 is to be performed on the input 218. The condition 208 of the function examines the input 218 for the results of Test 202 and determines if the results meet the condition 208. In this example, if the results for Test 202 are weak positive or positive, then the function 200 will perform action 210 to the input 218. The action 210 of the function 200 can involve considering combined outcomes of Test 202 and Test 204 and assigning a dynamic value. Performing the action 210 can involves applying the evaluation matrix 214.

The evaluation matrix 214 can include all possible combined outcomes subject to the condition 208 and a list of all possible dynamic values 212 for the function 200. In this example, six possible combined outcomes exist: three combined outcomes associated with a positive result for Test 202 (e.g., one each for positive, weak positive, and negative results for Test 204) and three combined outcomes associated with a weak positive result for Test 202 (e.g., one each for the positive, weak positive, and negative results for Test 204). Each of the six combined outcomes can have an associated dynamic value from among the dynamic values 212. The dynamic values 212 can include or describe a sentence, sentence fragment, multiple sentences, paragraphs, graph values, graphical elements like figures, and the like to be included in a customized report for a user of a CDS system, such as CDS system 100 described in FIG. 1. Dynamic values can also include instructions. For example, if a particular dynamic value becomes assigned, the assigned dynamic value can cause a second function to perform a set of actions. The instructions can also include instructions for an external device to perform an action, such as scheduling a future medical exam for the patient.

For example, a dynamic value associated with the combined outcome of both ‘weak positive’ results for Test 202 and Test 204 can include ‘Sentence 2’. Thus, the dynamic value for the combined outcome of two ‘weak positive’ results can describe a particular sentence to be included in the custom report. For example, the sentence could state: “Based on your recent test results, you should discuss additional testing with your primary care provider.” The dynamic value associated with the combined outcome of ‘weak positive’ for Test 202 and ‘positive’ for Test 204 can include ‘Sentence 3 and Sentence 2’. Therefore, the dynamic value for a combined outcome of ‘weak positive’ and ‘positive’ can include the same particular sentence described above with an additional sentence. For example, the sentences could state “Your recent test results indicate that you may have contracted disease A. Based on your recent test results, you should discuss additional testing with your primary care provider.” Dynamic values 212 of functions can be based on factors including expert opinions, recommendations in texts such as clinical care texts, results of surveys of medical professionals, feedback from users of the CDS system, and the like.

The function 200 can compare results in the input 218 to the possible combined outcomes in the evaluation matrix 214 and produce an assigned dynamic value 216 from among the possible dynamic values of the function 200. For example, the combined outcome described in the input 218 can include the ‘weak positive’ result for Test 202 and the ‘positive’ result for Test 204. Based on the results included in the input 218, the function 200 can investigate the evaluation matrix 214 and can designate “sentence 2+sentence 3’ as the assigned dynamic value 216 from among the dynamic values 212. The assigned dynamic value 216 can be the output of function 200 and the customized report for the user can include the assigned dynamic value 216 from function 200 as well as other assigned dynamic values from other functions of the CDS engine 108.

The evaluation matrix 214 of function 200 included six potential discrete combined outcomes consistent with the condition 208 of the function 200 and each combine outcome included a potential dynamic value. Other evaluation matrices can have a different number of combined outcomes and a different number of potential dynamic values. Some evaluation matrices can be formed based on a machine-learning model included in a function that can be trained to determine a likelihood that a patient has contracted a disease or medical condition based on data in the input to the function. The data in the input can include any number of tests, biographical information, family history, etc. for the patient. Training data for the machine-learning model can include historical data of many previous patients, medical records, autopsy records, etc. The likelihood can take on an array of continuous values from 0% (or not at all likely) to 100% (or certain). Ranges within the array can be associated with potential dynamic values. For example, if the likelihood is between 15% and 25%, the dynamic value can be Paragraph 1+Graph 1.

The entity can add, remove, and edit processes including functions for the CDS engine in an authoring environment. The entity can include administrators, computer scientists, programmers, medical department heads, laboratory directors, authorized users, etc. The entity can add or remove new functions, edit components of existing functions, update programming languages used to define functions, etc. The entity can also create and edit evaluation matrices of functions and the dynamic values associated with functions. Each dynamic value can include several lines of code or statements (e.g., CQL statements) in programming languages. In some examples, producing all necessary lines of codes to accurately describe all possible dynamic values for a set of medical scenarios can be a daunting task, consume valuable resources, and may lead to costly errors. For example, a field of next generation sequencing (NGS) can include millions, billions, or even trillions of possible combinations. Fully describing each combination using lines of code in computer programming may be a challenging task for the entity. In some cases, a tool of the authoring environment called an accelerator can be used to reduce an amount of resources required to create some functions, including, but not limited to, functions associated with genomic data.

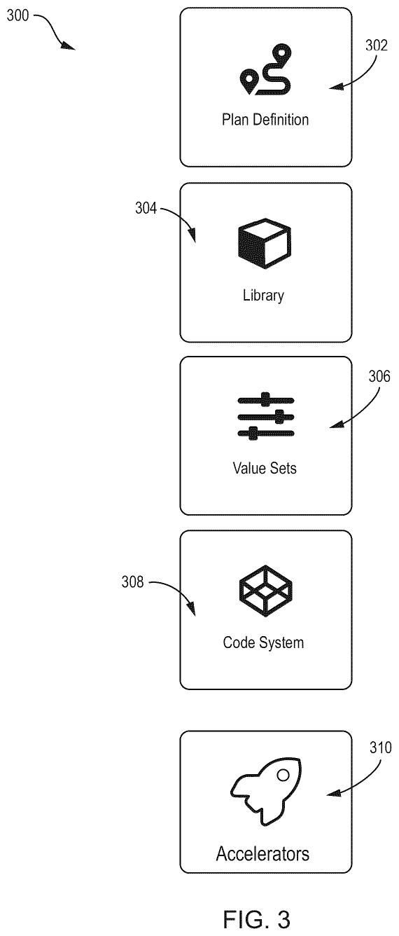

FIG. 3 is a diagram of graphical icons for tools or artifacts for a clinical authoring environment 300 for a CDS system (e.g., CDS system 100 of FIG. 1) according to some embodiments of the present disclosure. The graphical icons can be included in a GUI for the authoring environment 300. The clinical authoring environment 300 can include graphical icons for tools including plan definition 302, library 304, value sets 306, code system 308, and accelerators 310 graphical icons. The entity can access each tool by selecting an associated icon within the authoring environment 300.

The plan definition 302 graphical icon can provide a user access to a tool that can be used to create resources to navigate and orchestrate clinical decisions, care plans, or activities to produce a structured output for a consuming system of the CDS system. The library 304 graphical icon provides access to the library tool. The library tool can include rule logic, common functions, and linked software resources in a functional FHIR-compliant programming language called CQL. The value sets 306 graphical icon provides access to value sets. Value sets can include code groups for rule-specific and system-specific enumerators or references to allow the entity to manage included tests and clinical codes for any type of rule.

The code system 308 graphical icon provides access to a code system for the authoring environment 300. The code system can describe external and internal terminologies and other codable concepts that can be explicitly used in the clinical authoring environment 300. Both the internal and external terminologies can emphasize industry standard code systems but allow for custom records as well. Code systems can be a basis for value sets.

The accelerators 310 graphical icon can provide the entity with access to accelerators. Accelerators can be program and discipline-specific authoring spreadsheets. The authoring spreadsheets can describe rules and functions that can be imported and automatically converted into CQL code. Accelerators can allow for large, complex, and repetitive parameter sets, such as parameter sets including genomic data that may challenge an entity to program associated CQL code directly. Accelerators can also permit simple CSV file imports of third-party rule sets that can automatically be converted into CQL code.

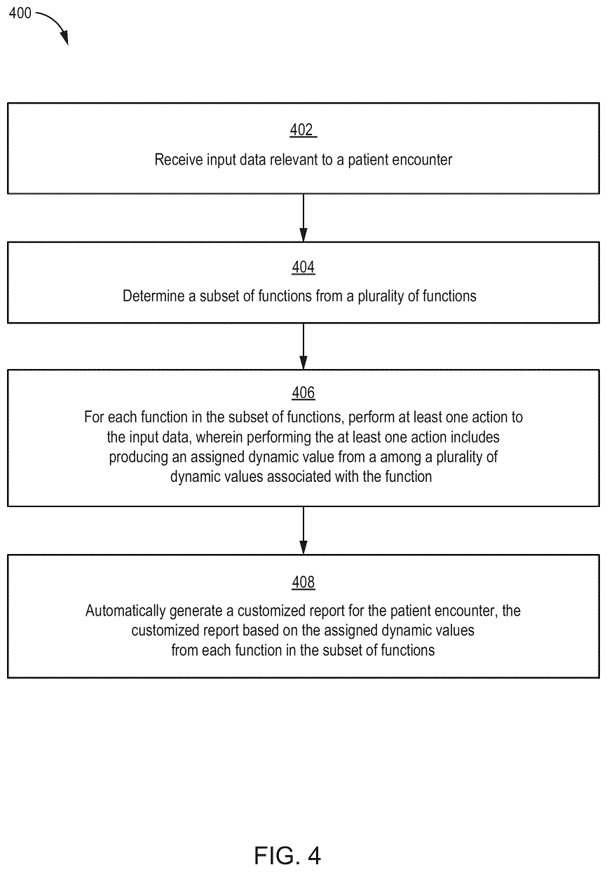

FIG. 4 is a flow chart of an example of a process 400 for automatically generating a customized report for a patient encounter according to some embodiments of the present disclosure. Other examples may include more operations, fewer operations, different operations, or a different sequence of operations than are shown in FIG. 4. The operations of the process 400 can start at block 402.

At block 402, the process 400 involves receiving input data relevant to a patient encounter. The input data can be compliant with a standard for exchanging healthcare information, such as FHIR. The input data can be received by a CDS system (e.g., the CDS system 100 described with respect to FIG. 1). The input data can include current data updated during the patient encounter, such as vital signs during the patient encounter including, but not limited to, a patient's body temperature, pulse rate, respiration rate, blood sugar level, glomerular filtration rate (GFR), urine albumin, or blood pressure. A patient identification service of the CDS system can use data relating to a patient involved in the patient encounter to identify the patient. For example, based on various personally identifiable information (PII), the patient identification service can identify the patient in addition to other data, such as data included in disjunctive sources, relevant to the patient. The input data can also include historical data related to the patient including data from medical records for the patient, including vital signs from previous patient encounters, diagnosed medical conditions, medications, billing data, observational data related to the patient, genome sequencing data, patient data, and physician data.

The observational data can include any combination of laboratory results including test results, test panel results, test chemistry results, analytic results, diagnostic procedure results, etc. Additionally, observational data can include various levels of details depending on types of tests performed and information being conveyed. For example, observational data can include laboratory results, physiological results, mental results, physical results, etc.

The data input can be received from a collection of databases. A record management system can aggregate and deduplicate data from the collection of databases to form the data input before transmitting the data input to the CDS system. The collection of databases can include data from multiple disjunctive sources. The multiple disjunctive sources can include a combination of electronic health record (EHR) systems, electronic medical record (EMR) systems, outpatient facilities (e.g., testing facilities), any medical facility visited by the patient, and any other suitable disjunctive sources.

The input data can be received as part of a request from a user via a consuming system. Examples of consuming systems can include a diagnostic assistant tool application, an application for evaluating potential clinical trials for the patient, application programming interfaces (APIs), patient portals, laboratory information management systems (LIMS), various interfaces, and the like. The input data can include data associated with the user or the consuming system. For example, the input data can identify the consuming system, or a medical field of specialization associated with the user.

In some examples, the input data can be prefiltered before a function performs any actions to the input data. For example, if a function receives a specific laboratory test result and the patient has received the laboratory test multiple times, the CDS system can reduce the input data associated with the laboratory test results into an average of the test results, into a number of most recent test results, into an average of a number of recent test results, into the most common result, etc.

At block 404, the process 400 involves determining a subset of functions from a plurality of functions. Functions can receive a portion of the input data as inputs, apply actions to the inputs including evaluating the inputs to produce an assigned dynamic value from among a group of potential dynamic values associated with the function, and provide outputs that can include the assigned dynamic value. The CDS system can include many functions. Some functions may not be relevant to the patient encounter. For example, functions that can evaluate and recommend prenatal preparations may not be especially relevant when a patient is a male. The CDS system can determine which subset of functions are relevant to the patient encounter based on the input data. A mapping service of the CDS system can map various data within the input data. The mapping service can map particular test results for the patient to particular medical conditions. The CDS system can identify functions associated with the particular medical conditions to include in the subset of functions.

In an example, the CDS system can compare health statistics for different patient encounters. A drastic change in weight can be detected for the patient by comparing a weight from the current patient encounter to a weight measured during a most recent previous patient encounter. For example, the patient may have lost thirty pounds (lbs), since the last patient encounter from six months ago. The CDS system can determine that the subset of functions can include any function that evaluates conditions that can be associated with the detected drastic change in weight. Determining the subset of functions can be based on previous diagnoses for the patient, laboratory results, family history, medications, vital signs, genomic data, such as genome sequencing data, other forms of data associated with health of the patient, etc.

The subset of functions can be determined based on parameters associated with the patient encounter, conditions met by the input data, or a consuming system that receives the customized report. Information associated with the request from the user via a consuming system can assist with determining the subset of functions. For example, the consuming system can be identified as the application for evaluating potential clinical trials for the patient and the CDS system can determine that the subset of functions can include any functions that evaluate a patient's qualifications for any open clinical trials. The user can be identified as an oncologist. The CDS system can identify oncology related functions to include in the subset of functions.

Each function can include metadata. The metadata can include a category for the function. The category can describe a disease, clinical group, etc. that is associated with the function. For example, the metadata for a function can indicate that the function is associated with a cardiovascular clinical group. Additionally, the metadata for a function can include at least one plan definition to associate the function with. The CDS system can use the metadata to help select which functions to include in the subset of functions. The subset of functions can include all functions associated with a plan definition, some functions associated with the plan definition, functions from multiple plan definitions, etc.

Functions can also include conditions to be met by data before the functions will perform an action to the data. Determining the subset of functions can be based on conditions of functions. The CDS system can analyze the data to see if conditions are met for a particular function and if the conditions are not met, the particular function may not be included in the subset of functions.

At block 406, the process 400 involves for each function in the subset of functions, performing at least one action to the input data. The at least one action can include producing an assigned dynamic value from among a group of dynamic values associated with the function. Dynamic values can include sentences, sentence fragments, multiple sentences, graph values, graphical elements, instructions, and the like. Prior to performing an action, the function can have many possible dynamic values. After performing the action, the function can have a single assigned dynamic value or several assigned dynamic values.

Dynamic values of functions can be based on factors including expert opinions, recommendations in texts such as clinical care texts, results of surveys of medical professionals, feedback from users of the CDS system, historical feedback from patients, and the like. For example, a function can indicate that based on patient data, the patient in the patient encounter has a serious condition such as cancer. An assigned dynamic value associated with the function can include tips for a medical practitioner for informing the patient of the condition in a manner that is honest but also can show empathy according to expert opinions. A group of medical experts can provide a list of possibilities for the tips including phrases, sentences, or paragraphs and the tips and the tips can be selected from among the possibilities. If repeated occurrences of a phrase, sentence, etc. emerge in the list, the repeated occurrences can be given more weight than other phrases, sentences, etc. when selecting the tips.

In producing assigned dynamic values, each function can apply logic, matrix logic, equations, medical principles, trained AI (or machine-learning (ML) models, sets of rules, etc. to the input data in order to determine results or outputs. Each function can produce several results and each result can be associated with a dynamic value. In some examples, possible dynamic values can be associated with several results or a range of results. For example, a function referred to as ‘Diabetes check’ can apply a trained ML model to patient data and determine a diabetes probability or likelihood that the patient has diabetes. The dynamic values for this example can include dynamic value A that is associated with a result of 0-20% for the diabetes probability, dynamic value B for a result in the range 20-40% for diabetes probability, dynamic value C for 40-60% diabetes probability, and so on. The Diabetes Check function can be applied to data and a result of 22% for the diabetes probability can be determined. For this result, the Diabetes Check function produces dynamic value B as an assigned dynamic value.

Functions can be components of a CDS engine. Each function in the CDS engine can be developed in an authoring environment. The authoring environment can include tools to assist the entity, such as computer programmers, in developing the functions. The functions can be developed using a CQL programming code. The tools can help reduce an amount of resources used to develop the functions. In the authoring environment, logic, matrices, equations, medical principles, trained or untrained AI (or machine-learning (ML) models, sets of rules, etc. can be programmed into each function. For some complex functions, programming rules, logic, etc. for such functions can be time consuming and can take thousands of lines of code to fully develop. Each possible result in a function can include many lines of programming code to develop and fully describe the result.

The authoring environment can include a library tool. From the library tool, the entity can access a database with pre-developed functions that the entity can incorporate in a development of a new function. Instead of writing many lines of code to develop the new function, the entity can combine and edit pre-developed functions, saving a considerable amount of time and labor. Additionally, the authoring environment can include an accelerators tool. The accelerators tool can provide the entity with access to accelerators.

Accelerators can be customized spreadsheets, such as program and discipline-specific authoring spreadsheets. The authoring spreadsheets can describe rules and functions that can be imported and automatically converted into CQL code. Accelerators can allow for large, complex, and repetitive parameter sets, such as parameter sets associated with genomic data including but not limited to data generated with NGS. Accelerators can also permit simple comma-separated values (CSV) file imports of third-party rule sets that can automatically be converted into CQL code. The authoring environment can also include a code system tool. The code system tool can include a real-time updated dictionary of terms, such as medical terms that can be used to automatically update the dynamic values of functions.

At block 408, the process 400 involves automatically generating a customized report for the patient encounter. The customized report can be based on assigned dynamic values from each function of the subset of functions. Information included in the customized report can be FHIR compliant. The customized report can be populated by the assigned dynamic values. Assigned dynamic values can include or describe a sentence, sentence fragment, multiple sentences, paragraphs, graph values, graphical elements like figures, and the like to be included in the customized report. A form of the customized report can depend on the consuming system. For example, when the consuming system is the diagnostic assistant tool application, automatically generating the customized report can involve automatically generating a graphical user interface (GUI) within the diagnostic assistant tool application and incorporating the customized report within the GUI.

The customized report can take the form of a portable document format (PDF) file, an application, an API, etc. In addition to generating customized reports, the CDS system can transmit instructions to a device based on FHIR outputs of the CDS engine. For example, the FHIR outputs can include a recommendation to order a particular lab test for the patient. The CDS system can instruct, based on assigned dynamic values included in the FHIR outputs, a device to automatically transmit an order request to an automized test ordering system to perform, schedule, or inquire about the particular lab test. Prior to transmitting the order request, a user of the CDS system or another medical practitioner can review and approve the order request. In some examples, the order request may be stored in memory for later retrieval. In addition, or alternatively, the results of the lab test may be stored in memory for later retrieval and/or for modifying the care plan of the patient further upon receiving the lab test results.

The customized report can be specified and customized for a disease, condition, medical department, clinical group, etc. The CDS system can identify a disease, condition, medical department, clinical group, etc. to associate the customized report with based on the metadata of functions in the subset of functions that perform actions on the data input. In some examples, the CDS system can identify more than one disease, condition, medical department, clinical group, etc. The CDS system via the CDS engine can generate several customized reports, one for each disease, condition, medical department, clinical group, etc. that is identified.

In a non-limiting example, a patient may visit a medical professional in order to undergo a physical health examination. The physical health examination may include tests related to, but not limited to, chronic kidney disease. The results of the tests may be the input data relevant to the patient encounter to be provided to the CDS engine. The CDS engine may receive the input data and may determine a subset of functions that may be relevant to the to the results of the tests. For example, the CDS engine may determine appropriate functions such as clinical and/or custom equations, value sets, and/or CQL statements to apply a transformation to the input data. For each function determined, the CDS engine may perform an action and/or transformation on the input data to produce an assigned dynamic value associated with the function. For chronic kidney disease, the CDS engine may receive the test results indicating a weak positive/positive result (e.g., as described above in regard to FIG. 2), and automatically output a customized report. The report may include the combined outcomes of the weak positive/positive results such as a sentence, “Your overall results indicate chronic kidney disease is possible. Your glomerular filtration rate (GFR) (a hallmark measurement of how well your kidneys filter blood) was weak-positive/positive. Talk to your healthcare professional to consider further testing. Confirmation of the diagnosis requires additional testing such as testing your urine albumin result (a hallmark measurement of the albumin protein found in your blood)”.

In some examples, customized dashboards, graphical user interfaces (e.g., GUI 500 as discussed later), and/or digital assistants may be generated based on the transformed input data. For example, customized dashboards, GUIs, and/or digital assistants may be generated for conditions related to, but not limited to, chronic kidney disease, cervical cancer, oncological conditions (e.g., leukemia, lung cancer, thyroid cancer, etc.), urological conditions, toxicology conditions, or combinations thereof. The customized dashboards, GUIs, and/or digital assistants may include the values calculated for various risks, suggestions for specific risks of the conditions, enhanced reporting logic, authoring environment suggestions and/or options, analysis testing results, analysis testing rules, analysis testing reports, patient handout materials, patient guides, or combinations thereof.

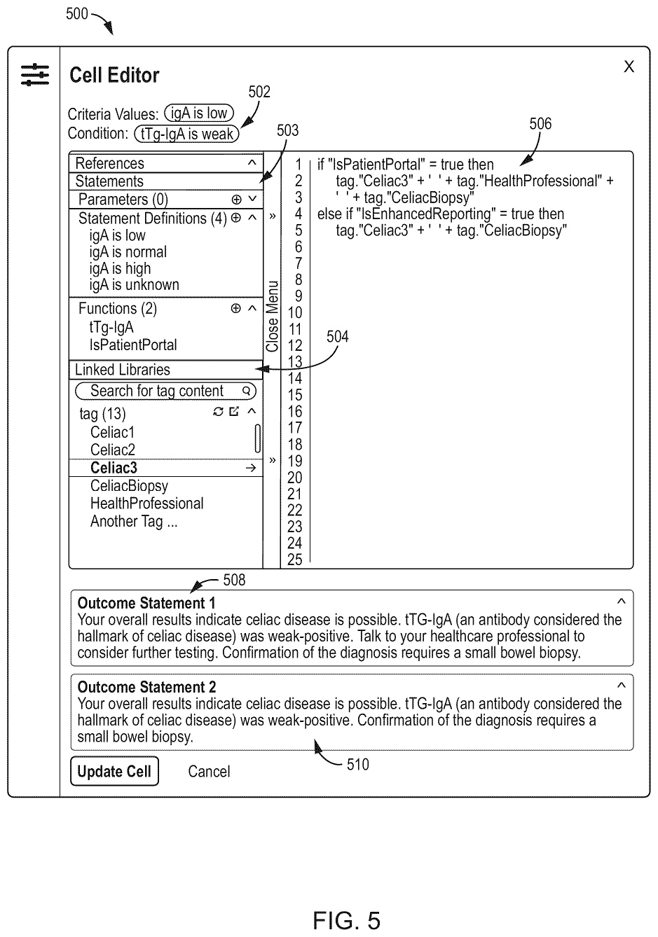

FIG. 5 is a depiction of a graphical user interface (GUI) 500 within an authoring environment for a CDS system according to some embodiments of the present disclosure. In the authoring environment, the entity can create, edit, or update components of a CDS engine of the CDS system. The components can also be referred to as functions or cells. The GUI 500 can include a condition 502, a statements portion 503 of the cell, a linked libraries portion 504 of the cell, a coding window 506, and two potential dynamic values: dynamic value 508 and dynamic value 510. Although two potential dynamic values are shown in GUI 500, components of the CDS engine can include any number of potential dynamic values including one dynamic value, zero dynamic values, millions of dynamic values, etc.

The condition 502 can define circumstances under which the cell or function will perform actions to input data. If the condition 502 is not met, the function may not perform an action or may not receive input data. The statements portion 503 of the cell can define a set of rules, logic, or nested logic that can guide a function in evaluating data and producing an assigned dynamic value based on input data. The linked libraries portion 504 of the cell illustrates that the entity can import previously created functions into the authoring environment, and significantly reduce the amount of time and labor involved in managing functions of the CDS engine. The coding window 506 allows the entity to view, edit, and create CQL coding for rules and functions.

Each function or cell of the CDS engine can also include dynamic values such as dynamic value 508 and dynamic value 510. The function can perform actions on input data, such as applying a trained ML model to the input data or applying a set of rules to the input data. Performing actions to the input data can also involve producing an assigned dynamic value from among a group of dynamic values associated with the function. In this example, the function can select either dynamic value 508 or dynamic value 510 as the assigned dynamic value to associate with the function. The selection of the assigned dynamic value can be based on results of actions performed to input data. The assigned dynamic value can be included as output for a patient encounter. A customized report can be automatically prepared based on assigned dynamic values and the customized report can be shared with a user of the CDS system.

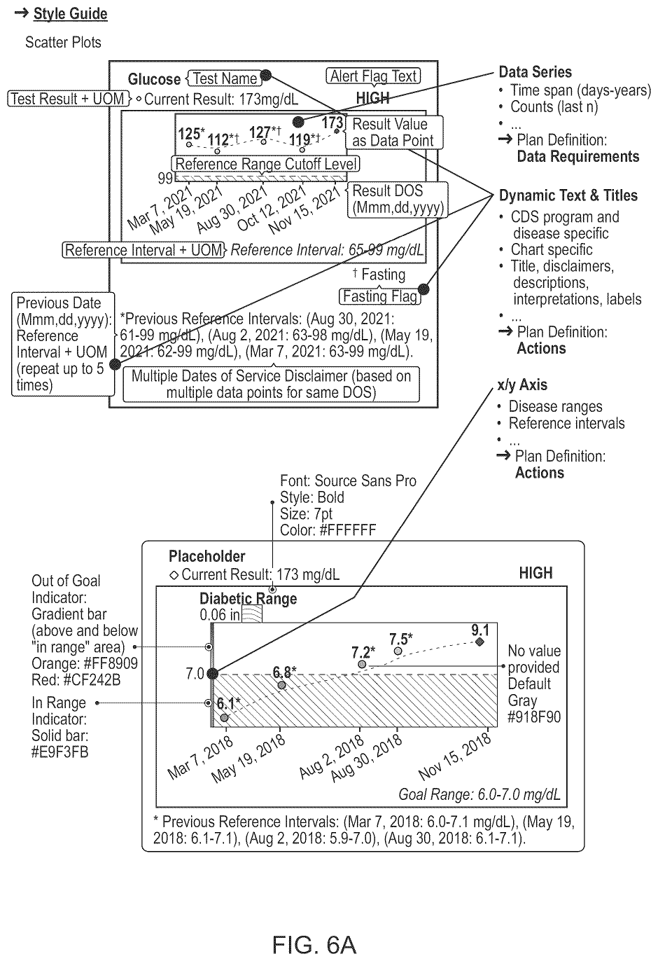

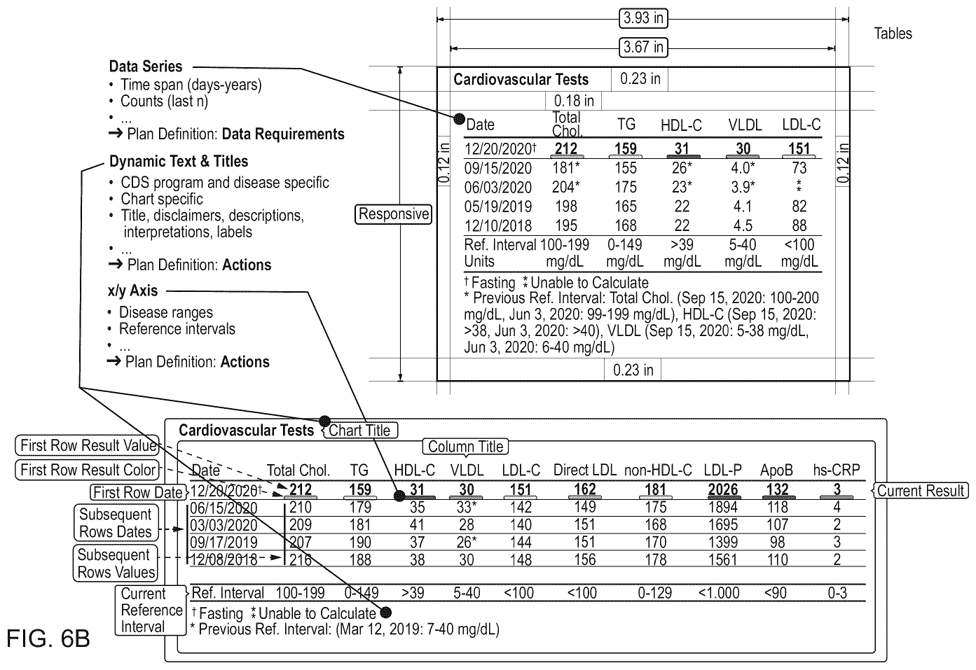

In a non-limiting example, a patient may visit a medical professional for cervical risk management. The patient may undergo one or more tests to find precancerous cervical cell changes. The results of these tests may be provided as input data to the CDS engine which may apply the functions to perform actions on the input data. For example, the CDS engine may apply one or more libraries to the input (e.g., identify which libraries are relevant to cervical cancer) to retrieve information related to cervical cancer. The information may include pre-determined statements which may be dynamically adjusted after-the-fact to provide a customized experience for the patient. In addition, or alternatively, the CDS engine may retrieve up-to-date peer reviewed literature on the subject of cervical cancer, value sets showing values related to cervical cancer. The CDS engine may combine the results of the functions applied to the input data according to code (e.g., such as code in coding window 506) to produce one or more dynamic values (e.g., dynamic values 508, 510). For example, if the patient's test results indicate a positive result of cervical cancer, the CDS engine may report the dynamic values in the GUI and provide a program for management to the patient and/or the medical professional. The program may be accessed by the patient remotely using a dashboard (e.g., some or all portions GUI as depicted in FIGS. 6A-6B) to provide ongoing care and treatment.

FIGS. 6A-6B are diagrams showing characteristics of customized reports generated by a CDS system according to some embodiments of the present disclosure. The CDS system can receive input data associated with a patient encounter. The CDS system can automatically generate the customized reports based on the input data. Before performing actions on the input data using a CDS engine, the CDS system can prefilter the input data to determine a portion of the input data that can be evaluated. The customized reports can be consistent with the prefilter process. For example, the CDS system can prefilter the input data and only perform actions on data from a specified time span such as over the past sixty days. Graphical elements of the customized reports can be consistent with the specified time span and include graphs with time axes that are confined to the last sixty days.

The customized reports can include dynamic text and titles. In some examples, the text and titles can be predetermined and CDS program or disease specific. Other portions of the customized reports can be program or disease specific as well, such as types of charts or graphs included in the customized reports, generated disclaimers, descriptions, explanations, interpretations, labels, etc. Graphs can also include disease specific details to help explain results. For example, if a particular enzyme amount or range of amounts can indicate a condition, a graph with enzyme readings can include a highlighted threshold value or range of values to help distinguish on the graph between normal readings that may not be indicators of the conditions, and abnormal readings that can be indicators of the condition.

Portions of the customized reports can be generated based on assigned dynamic values of components or functions of the CDS engine. Templates for the customized reports can be CDS program specific and assigned dynamic values can determine how each section of the template is populated by sentences, disclaimers, graphs, tables, etc. For example, if a trained machine-learning model is applied to the prefiltered data and determines that a likelihood that the patient has heart disease is above 50%, then an assigned dynamic value can indicate that the customized report includes a particular paragraph with an accompanying disclaimer, along with a graph to provide evidence for the condition, plus three sentences that explain how to interpret the graph and end, and footnotes.

In a non-limiting example, a patient may visit a medical professional for cancer screening (e.g., an oncology visit). The CDS system may retrieve input data (e.g., a serologic assay for a specific immunoglobulin (IgM)) from the patient and generate a customized report. The CDS system may prefilter the input data, such as identifying disease specific text and titles, specific charts, titles, disclaimers, interpretations, labels, or similar that are related to the input data. The customized report may include relevant data plots, tables, and/or dynamic text related to the patient specific input data. For example, for a positive test result for a specific type of cancer (e.g., identified biomarkers such as overexpressed proteins, mutated proteins, neoantigens, or similar), the CDS engine may output a tile containing a plot showing various ranges for a relevant disease, types of proteins, or similar. In some examples, the CDS engine may enable the patient and/or user of the CDS system to adaptively modify the plots, tiles, tables, axis, or similar to show and/or hide information related to the output. The output may be used, modified, and/or accessed by the patient and/or user using the GUI on-site and/or remotely. The output may include explanations and instructions for how to interpret the output. For example, if a specific protein biomarker is identified for leukemia, such as a nuclear speckle-related protein (e.g., NSRP70), information relating to the biomarker may be output to the patient as dynamic text such as, “Your overall results indicate leukemia is possible. A nuclear speckle-related protein, NSRP70, has been identified and is considered a hallmark of leukemia. Talk to your healthcare professional to consider further testing. Confirmation of the diagnosis requires additional testing and verification by your medical professional”, or, “Your overall results indicate leukemia is possible. A nuclear speckle-related protein, NSRP70, has been identified and is considered a hallmark of acute leukemia. NSRP70 enables mRNA binding activity and is involved in developmental processes and regulation of alternative mRNA splicing via spliceosomes and is located in the nuclear spec, and is a part of ribonucleoprotein complexes. Confirmation of the diagnosis will require additional testing by your medical professional at the earliest convenience”.

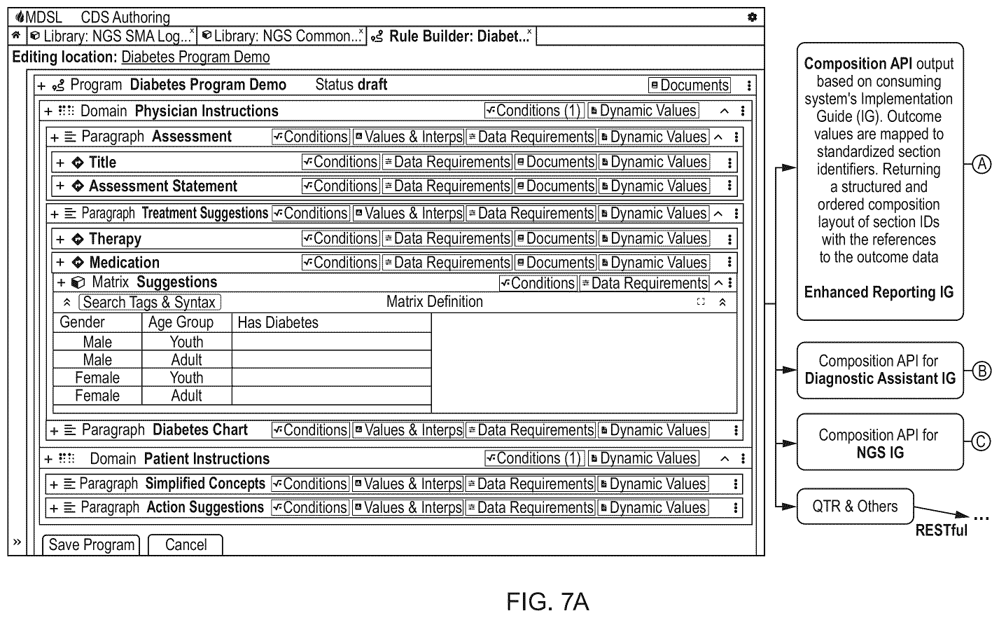

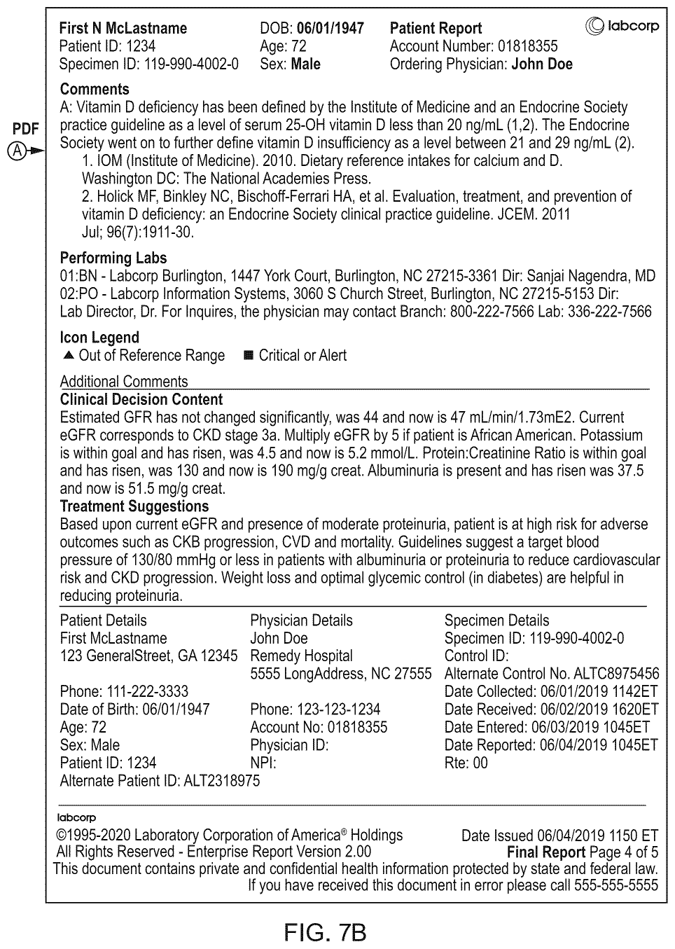

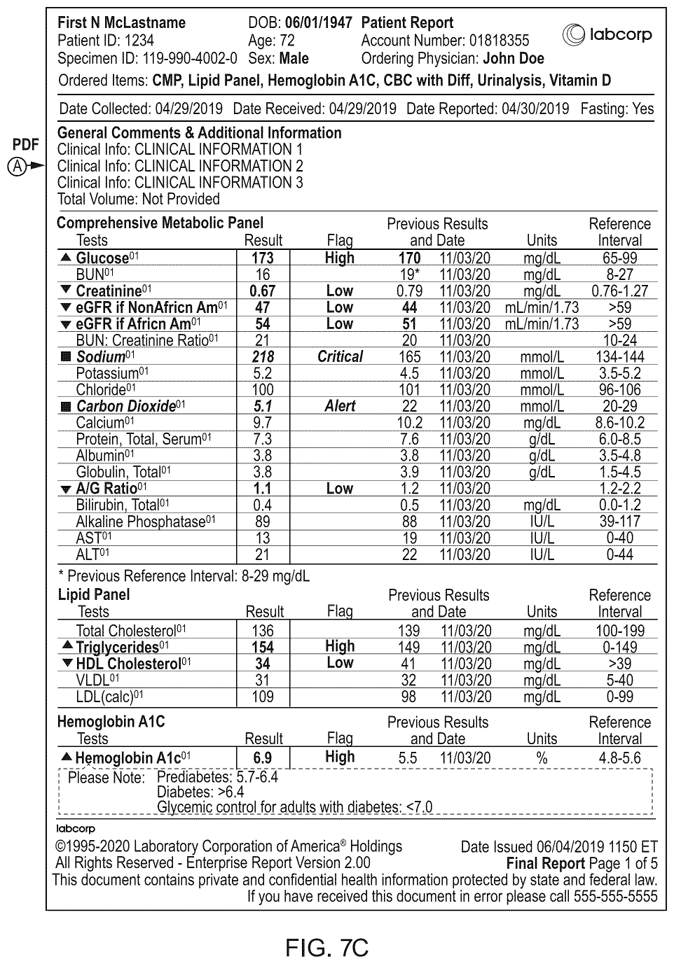

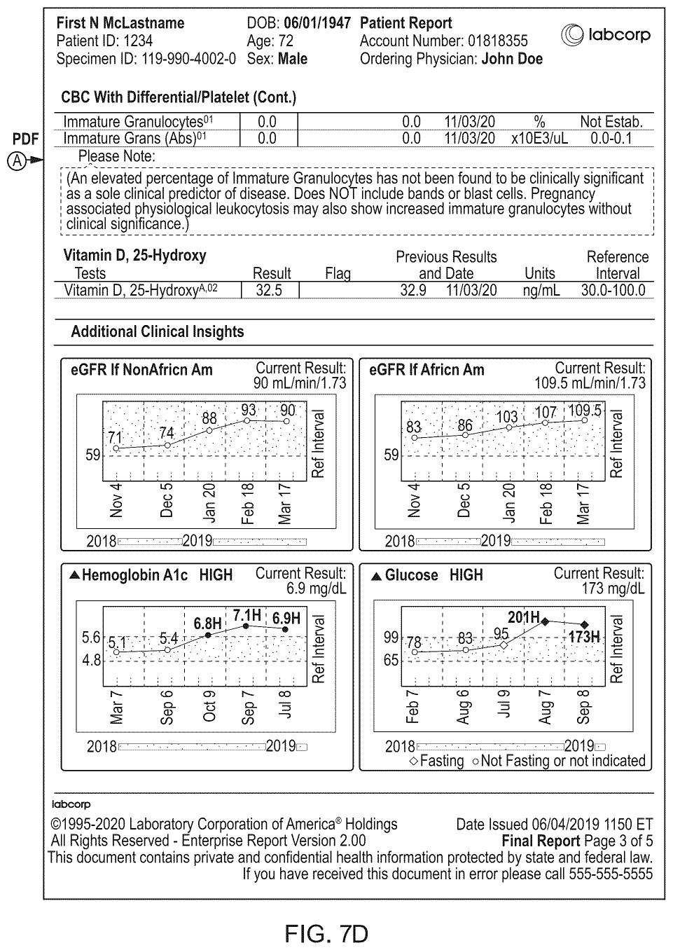

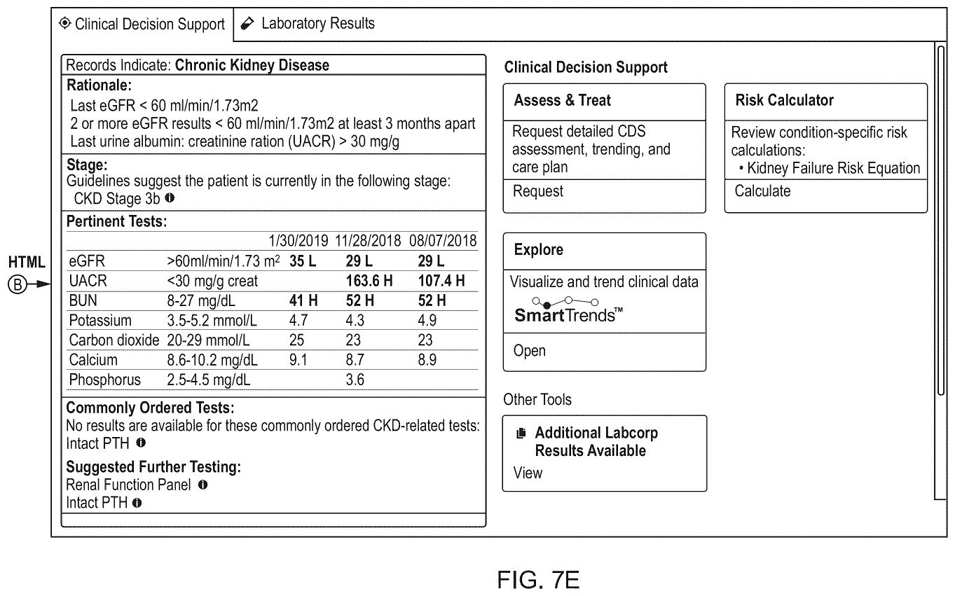

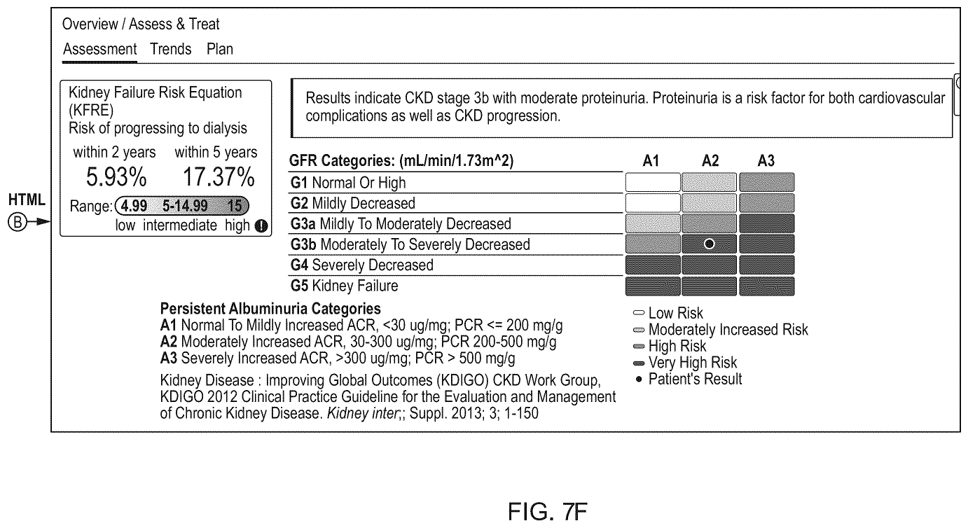

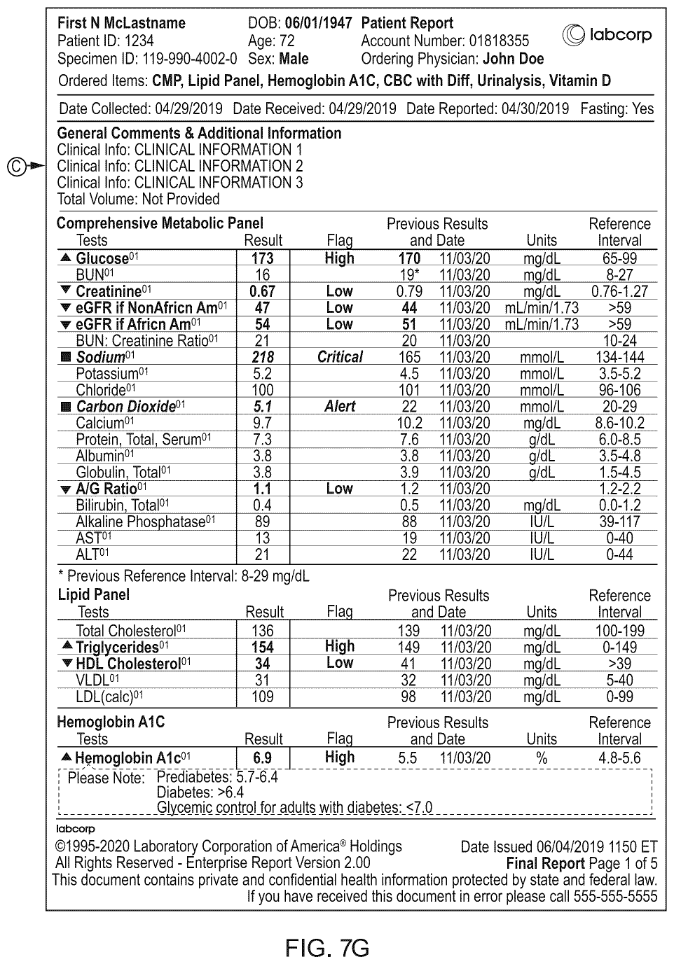

FIGS. 7A-7G are a schematic of customized reports tailored to consuming systems by a CDS system according to some embodiments of the present disclosure. Examples of consuming systems can include an enhanced reporting consuming system, a diagnostic tool application consuming system, an NGS consuming system, a quality time remaining (QTR) consuming system, etc. Each consuming system can include an implementation guide which can include implementation guidelines (IGs) for formats and templates for the customized reports.

Each IG can define a file type for the customized reports. For example, all customized reports for the enhanced reporting consuming system can be PDF files, whereas the customized reports for the diagnostic tool application can be in HTML form. Some customized reports can include a mixture of formats, such as a mixture of PDF and HTML formats. Outcome values determined by a CDS engine, such as assigned dynamic values, can be mapped to standardized section identifiers of the customized reports. The CDS system can include groups of composition Application Programming Interfaces (APIs) and each consuming system can be associated with one of the composition APIs. The composition APIs can return customized reports with a structured and ordered composition layout of section IDs with references to associated data.

In some examples, some specific conditions may have predesigned dashboards with associated programs/plans for treatment and care that may be adjusted by the medical professional and/or patient. For example, for patients diagnosed with chronic kidney disease (CKD), a patient may receive a report that includes a catered care plan for CKD, documents relating to CKD, a personalized care program that accounts for values associated with the patient (e.g., age, weight, blood pressure, time span, diet, etc.) and provide recommendations based on the values. In some examples, the dashboard may include the most common reports and/or API tiles (e.g., graphs, charts, tables, etc.) that are used for CKD. Other conditions that may include personalized dashboards, include, but are not limited to, A1C/Glucose diabetes management, kidney care management (e.g., twenty-four urine analysis protocols and reporting), oncology dashboards (e.g., LabCorp™ OminSeq, Myeloid), immune profiling dashboards, genomic profiling dashboards, or combinations thereof.

FIG. 8 is block diagram of a computing device 800 for use with the CDS system 100 in accordance with various embodiments. The computing device 800 includes a processor 805, which is in communication with the memory 810 and other components of the computing device 800 using one or more communications buses 815. The processor 805 is configured to execute processor-executable instructions stored in the memory 810 to perform one or more processes according to different examples, such as part or all of the example processes described above with respect to FIG. 4. In some examples, the memory 810 stores processor-executable instructions that provide the various components and their related functionality, as discussed above with respect to FIGS. 1-7. The computing device 800, for example, also includes one or more user input devices 830, such as a keyboard, mouse, touchscreen, microphone, etc., to accept user input. The computing device 800 also includes a display 835 to provide visual output to a user such as a user interface.

The computing device 800 also includes a communications interface 840. In some examples, the communications interface 840 may enable communications using one or more networks, including a local area network (“LAN”), a wide area network (“WAN”), such as the Internet, metropolitan area network (“MAN”), point-to-point or peer-to-peer connection, etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor includes a computer-readable medium, such as a random-access memory (RAM) coupled to the processor. The processor executes computer-executable program instructions stored in memory, such as executing one or more computer programs. Such processors may comprise a microprocessor, a digital signal processor (DSP), an algorithm-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, which may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

Although specific examples have been described, various modifications, alterations, alternative constructions, and equivalents are possible. Examples are not restricted to operation within certain specific data processing environments but are free to operate within a plurality of data processing environments. Additionally, although certain examples have been described using a particular series of transactions and steps, it should be apparent to those skilled in the art that this is not intended to be limiting. Although some flowcharts describe operations as a sequential process, many of the operations may be performed in parallel or concurrently. In addition, the order of the operations may be rearranged. A process may have additional steps not included in the figure. Various features and aspects of the above-described examples may be used individually or jointly.

Further, while certain examples have been described using a particular combination of hardware and software, it should be recognized that other combinations of hardware and software are also possible. Certain examples may be implemented only in hardware, or only in software, or using combinations thereof. The various processes described herein may be implemented on the same processor or different processors in any combination.

Where devices, systems, components or modules are described as being configured to perform certain operations or functions, such configuration may be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes may communicate using a variety of techniques including but not limited to conventional techniques for inter-process communications, and different pairs of processes may use different techniques, or the same pair of processes may use different techniques at different times.

Specific details are given in this disclosure to provide a thorough understanding of the examples. However, examples may be practiced without these specific details. For example, well-known circuits, processes, algorithms, structures, and techniques have been shown without unnecessary detail in order to avoid obscuring the examples. This description provides example examples only, and is not intended to limit the scope, applicability, or configuration of other examples. Rather, the preceding description of the examples will provide those skilled in the art with an enabling description for implementing various examples. Various changes may be made in the function and arrangement of elements.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific examples have been described, these are not intended to be limiting. Various modifications and equivalents are within the scope of the following claims.

In the foregoing specification, aspects of the disclosure are described with reference to specific examples thereof, but those skilled in the art will recognize that the disclosure is not limited thereto. Various features and aspects of the above-described disclosure may be used individually or jointly. Further, examples may be utilized in any number of environments and applications beyond those described herein without departing from the broader spirit and scope of the specification. The specification and drawings are, accordingly, to be regarded as illustrative rather than restrictive.

In the foregoing description, for the purposes of illustration, methods were described in a particular order. It should be appreciated that in alternate examples, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Where components are described as being configured to perform certain operations, such configuration may be accomplished, for example, by designing electronic circuits or other hardware to perform the operation, by programming programmable electronic circuits (e.g., microprocessors, or other suitable electronic circuits) to perform the operation, or any combination thereof.

While illustrative examples of the application have been described in detail herein, it is to be understood that the inventive concepts may be otherwise variously embodied and employed, and that the appended claims are intended to be construed to include such variations, except as limited by the prior art.

What is claimed is:

1. A method comprising:
receiving, by a computing device, input data relevant to a patient encounter, the input data compliant with a standard for exchanging healthcare information;
determining, by the computing device, a subset of functions from a plurality of functions based on parameters associated with the patient encounter, conditions met by the input data, or a consuming system that receives a customized report;
for each function in the subset of functions, performing, by the computing device, at least one transformation on the input data, wherein performing the at least one transformation comprises producing an assigned dynamic value from among a plurality of dynamic values associated with the function;
automatically generating, by the computing device, the customized report for the patient encounter based on the assigned dynamic values from each function in the subset of functions;
transmitting an order request, by the computing device, to order a lab test based, at least partially, on the customized report or the assigned dynamic values from each function in the subset of functions; and
in response to receiving a response to the order request, automatically transmitting, by the computing device, the order request to an automated test ordering system to perform the lab test.

2. The method of claim 1, wherein the consuming system is a diagnostic assistant tool application and wherein automatically generating the customized report comprises automatically generating a graphical user interface (GUI) within the diagnostic assistant tool application and incorporating the customized report within the GUI.

3. The method of claim 1, further comprising developing each function in the plurality of functions in an authoring environment.

4. The method of claim 3, wherein a portion of functions in the subset of functions is associated with at least one plan description defined in the authoring environment.

5. The method of claim 3, wherein developing each function in the plurality of functions comprises developing, in the authoring environment, each function to comprise sets of rules, logics, matrix logic, other common functions, artificial intelligence (AI) models, or value sets, wherein developing each function in the plurality of functions further comprises accessing, in the authoring environment, at least a portion of the sets of rules, logics, matrix logic, AI models, or value sets from a library.

6. The method of claim 3, further comprises developing, in the authoring environment, each function to comprise sets of rules, logics, matrix logic, other common functions, artificial intelligence (AI) models, or value sets.

7. The method of claim 3, wherein developing each function in the plurality of functions comprises developing each function using clinical quality language (CQL) programming code.

8. The method of claim 7, further comprising:
receiving, by the computing device, a customized spreadsheet for at least one function developed in the authoring environment; and
automatically converting, by the computing device, the customized spreadsheet into the CQL programming code.

9. A clinical decision support (CDS) system comprising:
one or more processors; and
one or more memories that include instructions executable by the one or more processors to perform operations comprising:
receiving input data relevant to a patient encounter, the input data compliant with a standard for exchanging healthcare information;
determining a subset of functions from a plurality of functions based on parameters associated with the patient encounter, conditions met by the input data, or a consuming system that received a customized report;
for each function in the subset of functions, performing at least one transformation on the input data, wherein performing the at least one transformation comprises producing an assigned dynamic value from among the plurality of dynamic values associated with the function;
automatically generating the customized report for the patient encounter based on the assigned dynamic values from each function in the subset of functions;
transmitting an order request to order a lab test based, at least partially, on the customized report or the assigned dynamic values from each function in the subset of functions; and
in response to receiving a response to the order request, automatically transmitting the order request to an automated test ordering system to perform the lab test.

10. The CDS system of claim 9, wherein the consuming system is a diagnostic assistant tool application and wherein the operation of automatically generating the customized report comprises automatically generating a graphical user interface (GUI) within the diagnostic assistant tool application and incorporating the customized report within the GUI.

11. The CDS system of claim 9, wherein the operations further comprise developing each function of the plurality of functions in an authoring environment.

12. The CDS system of claim 11, wherein a portion of functions in the subset of functions is associated with at least one plan description defined in the authoring environment.

13. The CDS system of claim 11, wherein the operation of developing each function in the plurality of functions comprises developing, in the authoring environment, each function to comprise sets of rules, logics, matrix logic, other common functions, artificial intelligence (AI) models, or value sets.

14. The CDS system of claim 13, wherein the operation of developing each function in the plurality of functions further comprises accessing, in the authoring environment, at least a portion of the sets of rules, logics, matrix logic, other common functions, AI models, or value sets from a library.

15. The CDS system of claim 11, wherein the operation of developing each function in the plurality of functions comprises developing each function using clinical quality language (CQL) programming code.

16. The CDS system of claim 15, wherein the operations further comprise:
receiving a customized spreadsheet for at least one function developed in the authoring environment; and
automatically converting the customized spreadsheet into the CQL programming code.

17. A non-transitory computer-readable medium comprising instructions that are executable by one or more processing devices for causing the one or more processing devices to perform operations comprising:
receiving input data relevant to a patient encounter, the input data compliant with a standard for exchanging healthcare information;
determining a subset of functions from a plurality of functions based on parameters associated with the patient encounter, conditions met by the input data, or a consuming system that received a customized report;
for each function in the subset of functions, performing at least one transformation on the input data, wherein performing the at least one transformation comprises producing an assigned dynamic value from among the plurality of dynamic values associated with the function;
automatically generating the customized report for the patient encounter based on the assigned dynamic values from each function in the subset of functions;
transmitting an order request to order a lab test based, at least partially, on the customized report or the assigned dynamic values from each function in the subset of functions; and
in response to receiving a response to the order request, automatically transmitting the order request to an automated test ordering system to perform the lab test.

18. The non-transitory computer-readable medium of claim 17, wherein the consuming system is a diagnostic assistant tool application and wherein the operation of automatically generating the customized report comprises automatically generating a graphical user interface (GUI) within the diagnostic assistant tool application and incorporating the customized report within the GUI.

19. The non-transitory computer-readable medium of claim 17, wherein the operations further comprise developing each function of the plurality of functions in an authoring environment.

20. The non-transitory computer-readable medium of claim 19, wherein a portion of functions in the subset of functions is associated with at least one plan description defined in the authoring environment.
